(19) United States
(12) Patent Application Publication
(10) Pub. No.: US 2008/0003281 A1
(43) Pub. Date: Jan. 3, 2008

(54) Modified Release Tablet Formulations for Proton Pump Inhibitors

(75) Inventors: Niclas Clemmensen, Molndal (SE); Jan-Erik Lofroth, Molndal (SE); Henrik Sahlen, Molndal (SE); Katrin Walter, Molndal (SE); Peter Wang, Molndal (SE); Martin Wikberg, Molndal (SE)

Correspondence Address: WHITE & CASE LLP;PATENT DEPARTMENT, 1155 AVENUE OF THE AMERICAS, NEW YORK, NY, 10036, US

(73) Assignee: ASTRAZENECA AB, Sodertalje (SE)

(21) Appl. No.: 11/718,571

(22) PCT Filed: Nov. 2, 2005
(86) PCT No.: PCT/SE05/01643

§ 371 Date: May 3, 2007

Related U.S. Application Data
(60) Provisional application No. 60/625,649, filed on Nov. 4, 2004.

(51) Int. Cl. A61K 31/4439; A61K 9/24; A61K 9/54; A61P 1/00

(52) U.S. Cl. 424/458; 424/472; 514/338

(57) ABSTRACT

An oral solid pharmaceutical dosage form comprising an acid sensitive proton pump inhibitor (PPI) as single active drug, releasing the PPI in two separate pulses, one immediate and one delayed. The PPI is formulated into a core material in the form of tablets, which are coated i.a. with a combination of a delay release modifying layer and a lag time controlling layer that together achieves beneficial release properties. The tablets are further provided with an enteric coating layer. The application also relates to processes for preparing the dosage forms as well as their use in the treatment of gastrointestinal diseases.

### FIELD OF THE INVENTION

This invention relates to an oral solid pharmaceutical dosage form comprising an acid sensitive proton pump inhibitor (including combinations of proton pump inhibitors) as only active drug in enteric coated delayed release tablets, as well as an improved process for their manufacture and the use of such dosage forms in medical treatment of gastrointestinal disorders.

### BACKGROUND OF THE INVENTION AND PRIOR ART

Acid sensitive H⁺, K⁺-ATPase inhibitors also named as gastric proton pump inhibitors are for instance compounds known under the generic names omeprazole, lansoprazole, pantoprazole, rabeprazole, leminoprazole and esomeprazole. Some of these compounds are disclosed in EP-A1-0005129, EP-A1-124495, WO 94/27988, EP-A1-174726, EP-A1-166287 and GB 2163747.

These pharmaceutical substances are useful for inhibiting gastric acid secretion in mammals including man by controlling gastric acid secretion at the final step of the acid secretory pathway and thus reduce basal and stimulated gastric acid secretion irrespective of stimulus. In a more general sense, they may be used for prevention and treatment of gastric-acid related diseases in mammals and man, including e.g. reflux oesophagitis, gastritis, duodenitis, gastric ulcer, duodenal ulcer and Zollinger-Ellison syndrom. Furthermore, they may be used for treatment of other gastrointestinal disorders where gastric acid inhibitory effect is desirable e.g. in patients on NSAID therapy, in patients with Non Ulcer Dyspepsia, and in patients with symptomatic gastro-oesophageal reflux disease (GORD). They may also be used in patients in intensive care situations, in patients with acute upper gastrointestinal bleeding, pre-and post-operatively to prevent aspiration of gastric acid and to prevent post-operative nausea and vomiting (PONV), and treat stress ulceration. Further, they may be useful in the treatment of psoriasis, sleep disturbance as well as in the treatment of Helicobacter infections and diseases related to these.

Enteric coated formulations comprising proton pump inhibitors (in the following also referred to as PPI's), and formulations intended to deliver PPI's after a delayed period of time have earlier been reported. However, currently available formulations of PPIs still have some shortcomings and limitations. The efficacy of acid control during PPI treatment is greater during daytime and after meals than during the night, which may have therapeutic consequences. A recent US study showed that nocturnal heartburn affects nearly 80% of individuals with GERD, resulting in sleep disturbance in 75% of these patients. The consequence of this is an impaired daily function in many patients (Shaker et al, AM J Gastroentrol 2003; 98 (7): 1487-93). Furthermore, there are some type of patients for which a more intensive gastric acid inhibition than the conventional once daily treatment might be needed. It has been shown that nocturnal gastric acid suppression can be significantly improved by splitting a 40 mg esomeprazole dose into 20 mg bid. This treatment regimen provides both rapid and sustained acid suppression (Hammer et al, Alimentary Pharmacol Ther 2004; 19 (19): 1105-10).

The present invention claiming an oral dosage form comprising two PPI releasing portions has been developed with the aim to securing an effective acid control over the whole 24-hour period, thus removing the necessity for twice daily dosing. This will provide an aid of use and patient compliance. Such a modified release formulation would also result in a greater efficacy in acid secretion inhibition, especially at night, compared with the conventional formulations of PPIs.

EP 247983 (AB Hassle) describes dosage forms of omeprazole or an alkaline salt of omeprazole wherein the active ingredient together with an alkaline reacting compound is formulated into a core material having a subcoating layer disposed thereon and an enteric coating as the outer layer. The dosage forms are intended to release the active ingredient rapidly in the small intestines after passage of the acidic milieu of the stomach

WO 9601623 and WO 9601624 describe tableted dosage forms of omeprazole and other proton pump inhibitors, wherein enteric coating layered pellets together with other excipients are compressed into a multiple unit tableted dosage form. It is essential in these table ted formulations that the enteric coating layer can withstand the compression forces during tabletting.

WO 9932093 A1 (Astra AB) discloses an enteric coated pharmaceutical dosage form comprising an H⁺,K⁺-ATPase inhibitor. The formulation comprises at least two portions of the H⁺,K⁺-ATPase inhibitor to be released in at least two consecutive pulses. At least one of the portion has a delayed release. Those pellets or tablets giving the delayed release pulse include a surrounding lag time controlling layer, which is a semipermeable membrane comprising a water resistant polymer, and which disrupts after a desired time. There is no disclosure of a combination of a delay release modifying layer and a lag time controlling layer wherein the latter consists mainly of a high viscosity water soluble polymer.

U.S. Pat. No. 5,885,616 (Impax Pharmaceuticals Inc.) discloses a single bead drug delivery system which can provide a two-step release of active agent to facilitate an immediate yet sustained drug delivery. It does not disclose a lag time controlling layer comprising a high viscosity water soluble polymer as the only or the essential polymer. Neither does it disclose or suggest this delivery system for PPI's.

WO 9819668 (Sharmatek) is directed to a multicompartment delayed release drug delivery system for acid sensitive drugs like omeprazole. The delayed release is related to a delayed release enteric barrier providing gastro-resistant behaviour for delivering omeprazole in the proximal segment (pH 5-6) of the gastrointestinal tract. This enteric barrier comprises enteric coating polymers as material of this layer. There is no disclosure of a high viscosity water soluble polymer.

EP 1194131 B1 (Sanofi-Synthélabo) discloses a controlled release dosage form producing at least a timed pulse. The delayed release is achieved with a coating comprising one or more ammonio methacrylate copolymers (water insoluble polymers). The drug may be omeprazole. It does not disclose a lag time controlling layer comprising a high viscosity water soluble polymers as the only or the essential polymer. Neither does it disclose any delay release modifying layer according to the invention in the present application, nor any enteric coating layer.

WO 0158433 (Eurand) discloses a pharmaceutical dosage form such as a capsule, comprising a multitude of multicoated particulates as beads, pellets or granules. If the beads are not immediate release beads they have at least two coated membrane barriers. One of them is composed of an enteric polymer while the second membrane barrier is composed of a mixture of a water insoluble polymer and an enteric polymer. Further, they also have an optional intermediate membrane containing an acid. It does not disclose a lag time controlling layer comprising a high viscosity water soluble polymer as the only or the essential polymer. Neither does it disclose or suggest this delivery system for PPI's.

WO 0124777 (American Home Products) discloses a pharmaceutical formulation for once daily administration providing a phased release of a drug or particularly multiphase delivery of PPI's such as perprazole (nowadays known as Esomeprazole). The core is surrounded by an outer semi-permeable membrane comprising a permeable water insoluble polymer and at least 50% by weight of glidant. The units lack an enteric coat. The patent application does not disclose a lag time controlling layer comprising a high viscosity water soluble polymer as the only or the essential polymer.

U.S. Pat. No. 6,749,867 B (Robinson, J. R. and McGinity, J. W.) presents a time-release dosage form for acid-sensitive drugs or more particularly omeprazole, including a drug-containing core surrounded by an inert time-release coating, being water soluble or water erodible, delaying release to generally 0.5-5.0 hours after administration. The formulation has no enteric coat.

WO 2000078293 A1 (AstraZeneca AB) presents a dosage form for omeprazole or an alkaline salt thereof, S-omeprazole or an alkaline salt thereof, as active ingredient in a core together with alkaline additive(s) and swelling agent(s). The core is coated with a semipermeable membrane, achieving a delayed release starting when the membrane disrupts. The polymers disclosed for use in the semipermeable membrane are water insoluble polymers. The formulations have no enteric coat.

EP 1086 694 A2 (Laboratorios Del Dr. Esteve, S. A.) presents a solid oral pharmaceutical formulation for acid sensitive benzimidazoles in the form of pellets. The pellets have at least a system for modified release that achieve slow release profiles by an intermediate layer comprising a combination of an inert, noalkaline polymer insoluble in water (ethylcellulose) and an inert, nonalkaline polymer soluble in water (hydroxypropyl methyl cellulose). The slow release pellets can be mixed with fast release pellets and formulated into capsules or tablets.

WO 2002053097 A2 (Tap Pharmaceutical Products, Inc. USA) presents a nonenteric coated carrier for a proton pump inhibitor, including a bicarbonate or a carbonate salt of a Group IA metal.

None of these previously described formulations disclosed a dosage form having a combination of a delay release modifying layer and a lag time controlling layer, the latter comprising a high viscosity water soluble polymer or discloses a dosage form having a dissolution pattern as described in this patent application.

There is still a need for a dosage form comprising an acid sensitive proton pump inhibitor (PPI) in which formulation the PPI can be transported intact through the stomach and then after a further desired delay time the dose of the PPI will be rapidly released, together with a PPI portion that is rapidly released directly after the passage of the stomach, without any further delay time.

One way to produce such formulations is to construct them as small layered tablets.

Manufacturing processes for coating layered tablets comprise most frequently some type of spraying process. Problems experienced with this technique, especially when spraying a solution of a high viscosity hydrophilic polymer, is that the processing times are often too long for practical use.

### BRIEF DESCRIPTION OF THE INVENTION

The invention relates in one aspect to an oral solid pharmaceutical dosage form comprising as the single active drug an acid sensitive proton pump inhibitor (PPI) comprised in a core material in the form of small tablets, which tablets are comprised in said dosage form giving release with a delayed release pulse and an immediate release pulse, and in which the tablets having delayed release accomplish the delayed release effect by that these tablets have the following layers on the core material in the given order; a delay release modifying layer, a lag time controlling layer comprising as essential component a high viscosity water soluble polymer, an optional subcoating layer, and an outer enteric coating layer; and in which dosage form said tablets are comprised together with a portion of pellets or tablets giving immediate release of the PPI.

The immediate release is achieved as described earlier in the art, as immediate release enteric coated pellets or tablets.

In the invention, the small tablets are smaller or equal to 5 mm in diameter, and when the small tablets are assymetrical, their longest axis is smaller or equal to 5 mm.

In a second aspect of the invention the oral solid pharmaceutical dosage form is comprising as the single active drug an acid sensitive proton pump inhibitor (PPI), comprised in a core material in the form of tablets, which tablets are comprised in the dosage form giving release with a delayed release pulse and an immediate release pulse, and in which the tablets having delayed release and immediate release accomplish these effects by having the following layers on the core material in the given order; a delay release modifying layer, a lag time controlling layer comprising as essential component a high viscosity water soluble polymer, a layer comprising a 2^{nd} PPI portion giving immediate release, and an outer enteric coating layer optionally preceeded by a subcoating layer.

The finalized dosage forms of the invention comprise as one element an immediate release portion (releasing the drug immediately after passing of the acidic milieu of the stomach) and as a second element a delayed release drug portion, which after first passing the acidic milieu of the stomach and then is released after a further lag time (with negligable release) which is being in the range of 1-10 hours.

It has now surprisingly been found that the dosage forms of the invention have improved dissolution characteristics. These are that besides having a further delay (besides the one resulting from the enteric coating) the dissolution of the delayed pulse may be more distinct than in prior art. This have been found to be an attribute of the combined delay release modifying layer and lag-time controlling layer.

This more distinct dissolution effect may be seen as an increased steepness for the dissolution curve for the delayed pulse once the dissolution commences.

The acid sensitive proton pump inhibitors are formulated into tablet cores according to conventional methods, together with pharmaceutically acceptable excipients.

The tablet cores are coated with a delay release modifying coating layer before applying the lag-time controlling coating layer.

This is accomplished by a further aspect of the invention, being a new inventive process for applying the lag-time controlling layer, in which process cores comprising an acid sensitive proton pump inhibitor as single active ingredient (and coated with the delay release modifying layer) are coated with a high viscosity water soluble polymer (like e.g. hydroxypropyl methyl cellulose, also referred to as HPMC in the following, 4000 cps), in a dispersion. Using a dispersion of the high viscosity water soluble polymer makes the process advantageous in aspects like possibility of using higher concentration when spraying in a continuous mode, i.e. higher than compared with solutions, and possibility of using a higher spraying rate thereby giving a reduced processing time. This makes the process more simple, industrially more attractive and more economic than existing spraying techniques for these types of polymers.

Reported problems like clogging are also avoided, and thus there is a reduced need for addition of extra additives, e.g. anti-tacking agents.

Another advantage obtained with the new process is the improved release characteristic of the acid sensitive proton pump inhibitor from the products having the combination of a delay release modifying layer and a lag time controlling coat applied on the cores before the outer enteric coating is applied.

A third aspect of the invention is to use an alkaline quality of the high viscosity water soluble polymer in the lag time controlling layer, such as e.g. hydroxypropyl methyl cellulose or of hydroxyethyl cellulose (the latter also referred to as HEC in the following). This gives i.a. stability advantages.

A double pulse dissolution is achieved either by mixing of the enteric coated delayed pulsed release tablets with enteric coated instant/immediate releasing pellets/tablets according to the art (e.g. as described in EP 247983 , WO 9601623 or WO 9601624), and filling them into capsules or sachets, or incorporating the mixture together with excipients into a tablet by compression, or by coating the lag-time coated cores with a further (second) fast releasing/dissolving layer comprising the acid sensitive proton pump inhibitor as single active drug, before coating with an enteric coat, optionally preceded by a subcoating after the second drug layer.

The layer applied on a tablet core material comprising a second drug portion is according to the invention comprising disintegrants, e.g. Croscarmellose sodium.

Doses foreseen to be used in the double pulsed embodiment of the invention is in the range of 2-500 mg divided into an immediate release portion and a delayed release is portion of the acid sensitive proton pump inhibitor, suitably in combinations of e.g. equal doses e.g. 60 mg+60 mg, but doses divided into variable proportions are also contemplated, like e.g. 40 mg+120 mg.

Doses foreseen, for the single delayed release pulse formulation embodiment, being comprised in the final preparation, are in the range of 1-400 mg.

The dosage forms are advantageously used to provide a method of treatment for Crohn's disease, acute bleeding, ulcerous colitis, gastric ulcers, duodenal ulcers, gastroesoephagal reflux disease and the other diseases mentioned above.

### BRIEF DESCRIPTION OF THE DRAWINGS

FIG. 1 illustrates some of the definitions used in this application. See also the text in the part “Definitions” before the Examples.

FIG. 2 illustrates the release profile obtained from 6 individual units of the embodiment according to Example 1.

FIG. 3 illustrates the release profile obtained from Example 4. The * on the x-axis designates that 0-100% released is considering the initial dose of PPI and that the range 100-200% is considering 100% of the delayed (second) dose of PPI.

### DETAILED DESCRIPTION OF THE INVENTION

The dosage forms of the invention comprise an acid sensitive proton pump inhibitor (in the following also referred to as PPI) as the only active drug.

In one special embodiment of the invention, the PPI in the immediate release pulse is another one than the PPI in the delayed release pulse. Still this dosage form comprises only PPI's as active drug.

These drugs are compounds of the general formula I, an alkaline salt thereof, one of the single enantiomers thereof or an alkaline salt of one of the enantiomers 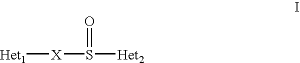
wherein

Het₁ is 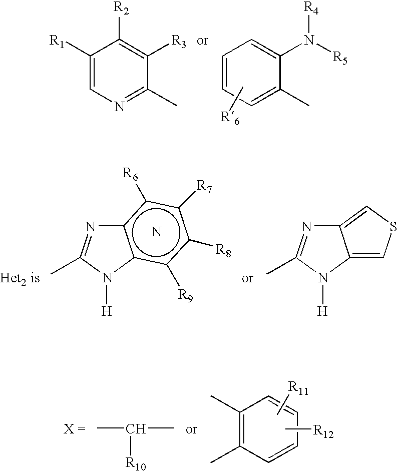
wherein

N in the benzimidazole moiety means that one of the ring carbon atoms substituted by R₆-R₉ optionally may be exchanged for a nitrogen atom without any substituents;

R₁, R₂ and R₃ are the same or different and selected from hydrogen, alkyl, alkoxy optionally substituted by fluorine, alkylthio, alkoxyalkoxy, dialkylamino, piperidino, morpholino, halogen, phenyl and phenylalkoxy;

R₄ and R₅ are the same or different and selected from hydrogen, alkyl and arylalkyl;

R₆′ is hydrogen, halogen, trifluoromethyl, alkyl or alkoxy;

R₆-R₉ are the same or different and selected from hydrogen, alkyl, alkoxy, halogen, halo-alkoxy, alkylcarbonyl, alkoxycarbonyl, oxazolinyl, and trifluoroalkyl, or adjacent groups R₆-R₉ form ring structures which may be further substituted;

R₁₀ is hydrogen or forms an alkylene chain together with R₃ and

R₁₁ and R₁₂ are the same or different and selected from hydrogen, halogen or alkyl.

In the above definitions alkyl groups, alkoxy groups, and moieties thereof may be branched or straight C₁-C₉-chains or comprise cyclic alkyl groups, for example cycloalkylalkyl.

Examples of specifically interesting compounds according to formula I are

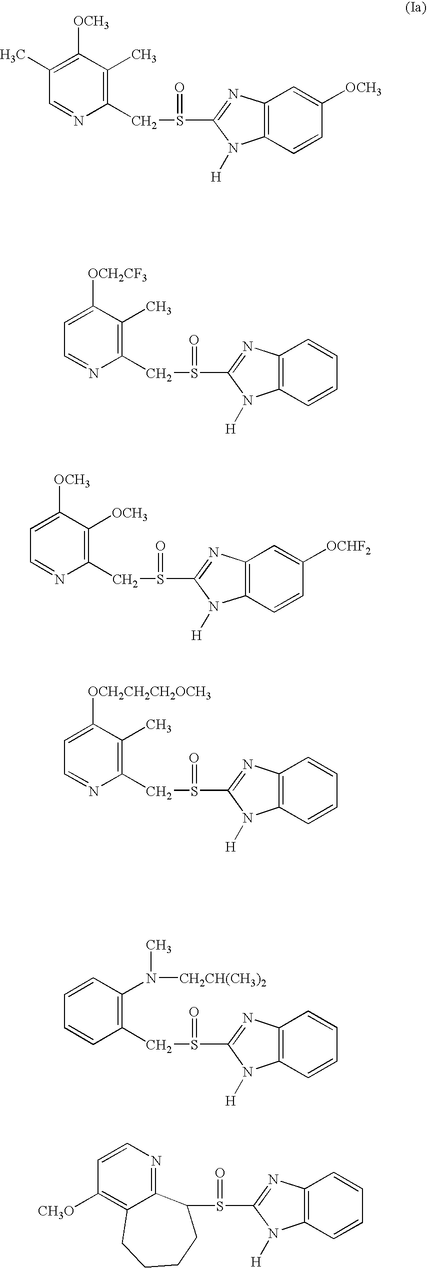

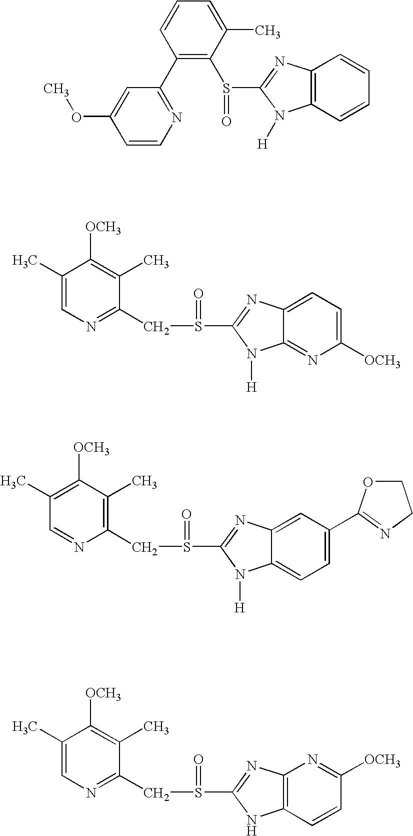

Preferred compounds for the oral pharmaceutical preparation according to the present invention are omeprazole, a magnesium salt of omeprazole or a magnesium salt of the (−)-enantiomer of omeprazole. The latter, the (−)-enantiomer of omeprazole, being named esomeprazole.

Especially preferred is an alkaline salt of esomeprazole, and most especially preferred is esomeprazole magnesium trihydrate.

In another embodiment of the invention tenatoprazole or one of its single enantiomers or an alkaline salt thereof, or an alkaline salt of tenatoprazole, is the active drug.

In a further, special embodiment of the invention tenatoprazole or one of its single enantiomers or an alkaline salt thereof, or an alkaline salt of tenatoprazole, is the active drug in one pulse and another PPI is the active drug in the other pulse.

Doses

Doses foreseen to be used in the used double pulsed embodiment of the invention is in the range of 2-500 mg divided into one immediate release portion and one delayed release portion of the acid sensitive PPI, suitably in combinations of e.g. equal doses e.g. 60 mg+60 mg.

The invention also provides doses divided into variable proportions, like dividing the dose in proportions being 20%+80% of the total dose in one contemplated specific embodiment, in proportions being 30%+70% of the total dose in a 2nd contemplated specific embodiment and even further in proportions being 40%+60% in a third contemplated specific embodiment, without excluding any other possible dividing ratio between the immediate portion and the delayed release portion.

Doses foreseen, for the single delayed release pulse formulation embodiment, being comprised in the final preparation, are in the range of 1-400 mg. Preferably the dose is 2-200 mg, and most preferably the dose is 5-120 mg.

Tablet Cores

The acid sensitive proton pump inhibitor comprising cores are formulated of the active drug optionally together with pharmaceutically acceptable excipients into a core material in the form of small tablets, smaller or equal to 5 mm in diameter, according to conventional methods. When the small tablets are assymetrical, their longest axis is smaller or equal to 5 mm.

Among excipients in the cores may be mentioned (without restricting them to); diluents/fillers, lubricants/glidants, pH regulating additives, disintegrants, osmotic agents, binders etc.

In a preferred embodiment the cores are exempt of acidic compounds. Acidic compounds according to this invention are compounds that give a pH of 5 or lower when dissolved or suspended in purified water at a concentration of 10% w/w (at room temperature, i.e. approx. 20 degrees Celsius), and measured with pH-meter equipped with a glass electrode or ISFET electrode.

The cores may be made by direct compression of active substance and excipients, alternatively after granulation procedures involving active substance and/or excipients. Any suitable granulation procedure known in the art, such as wet granulation, dry granulation or melt granulation, may be utilized.

The powders/particles are if needed conditioned to obtain a low moisture content, e.g. by drying in drying cabinets or/and by use of vacuum. Preferably, the moisture content after drying of the granulated powders/particles is less than 2% w/w.

If needed, the granulated particles are milled to reduce the particle size distribution and to obtain a powder mass with good flow properties.

In a preferred embodiment of the invention, granulated particles are sieved to pass a sieve having 1.0 mm openings.

Powders/granulations intended for compression into tablets may need additives like lubricants, glidants and disintegrants to be admixed before compression.

Such additives include but are not limited to; Mg-stearate, sodium stearylfumarate, glyceryl behenate, talc, fumed silica (E.g. Aerosil® and Cab-O-Sil®), sodium starch glycolate, microcrystalline cellulose, cross-linked sodium carboxymethyl cellulose (Cros-Carmellose sodium) and cross-linked polyvinyl pyrrolidone.

Compression is preferably performed with circular punches, but other shapes are not excluded. In the invention the tablet cores are compressed into small sizes, having diameters smaller or equal to 5 mm, preferably in the range of 0.5-5 mm. When the small tablets are assymetrical, their longest axis is smaller or equal to 5 mm, preferably in the range of 0.5-5 mm.

In one embodiment of the invention the tablet core is round and the diameter is in the range of 0.5-3 mm.

A suitable compression force is applied to obtain tablet cores that have the desired hardness necessary for the following coating operations, while they at the same time are having an acceptable disintegration time.

Delay Release Modifying Layer

The delay release modifying layer that is applied onto the core material, and separates the lag time controlling layer from the PPI containing core is hydrophobized by incorporation of a hydrophobizing agent and talc in a water soluble polymer based layer.

Thus, the delay release modifying layer comprises a water soluble polymer(s), talc and a hydrophobizing agent which e.g. can be selected from the group consisting of Mg-stearate, glyceryl behenate and sodium stearyl fumarate.

Water soluble polymers in the delay release modifying layer are chosen to be solid polymers and have a viscosity below 180 mPas (cps) tested according to the European Pharmacopoeia. Also mixtures of such polymers are contemplated for use in the invention.

It is also important the delay release modifying layer does not include compounds having free acidic groups such as carboxylic acid groups or sulphonic acid groups in its composition, such as e.g. carbomers or enteric coating polymers. Thus, the release modifying layer is free from compounds having one or more free acidic group(s).

Examples of water soluble polymers to be used include; Hydroxypropyl cellulose, hydroxypropyl methyl cellulose, polyethylene glycol, polyvinyl alcohol, polyvinyl pyrrolidone, polyethylene-polypropylene glycol copolymers and the like. The ratio between the water soluble polymer and talc is in the range of 1:1 to 1:8 (w/w), preferably in the range of 1:2 to 1:6 (w/w), and most preferably in the range of 1:3 to 1:4 (w/w).

The ratio between the water soluble polymer and the hydrophobizing compound is in the range of 3:1 to 5:1 (w/w), preferably 3.5:1 to 4.5:1 (w/w).

When the water soluble polymer in the delay release modifying layer is chosen to be hydroxypropyl cellulose (in the following also referred to as HPC), it is having a hydroxypropyl content in the range of 50-90% or more preferably in the range of 60-81%, and a viscosity below 180 mPas (cps) tested at 5% concentration. Such a polymer is, example given, Klucel LF, from Aqualon.

The hydroxypropyl celluloses contemplated for use in this aspect of the invention, as a water soluble polymer in the delay release modifying layer, do not include Low-substituted hydroxypropyl cellulose, also referred to as L-HPC.

In a preferred embodiment of the invention the hydrophobizing agent is selected from the group consisting of Mg-stearate, glyceryl behenate and sodium steryl fumarate, or from mixtures thereof.

In one specific embodiment of the invention the watersoluble polymer is hydroxypropyl cellulose and the hydrophobizing compound is Mg-stearate.

In this embodiment of the invention the delay release modifying layer is only composed of the three excipients hydroxypropyl cellulose, talc and Mg-stearate, disregarding minor traces of solvents/water that may be remains from the coating process.

In this specific embodiment the ratio between HPC and talc is in the range of 1:1 to 1:8 (w/w), preferably in the range of 1:2 to 1:6 (w/w), and most preferably in the range of 1:3 to 1:4 (w/w).

Further, in the same specific embodiment the ratio between HPC and Mg-stearate is in the range of 3:1 to 5:1 (w/w), preferably 3.5:1 to 4.5:1 (w/w).

In an alternative specific embodiment of the invention the watersoluble polymer is hydroxypropyl cellulose and the hydrophobizing compound is sodium stearyl fumarate.

Lag Time Controlling Layer

The lag time controlling layer comprises a high viscosity water soluble polymer like e.g. hydroxypropylmethylcellulose 4000, as essential component. The term “a water soluble polymer” as used herein means a water soluble polymer, water soluble copolymer, or mixture of such polymers. With high viscosity in this invention is regarded an apparent viscosity of 100 mPas (cps) up to 15 000 mPas (cps), tested according to as first alternative the European Pharmacopoeia and as second alternative the US Pharmacopoeia. In case of that tests are described in both pharmacopoeias, the method in the European one has prevalence.

In an alternative embodiment of this invention, the term high viscosity is regarding an apparent viscosity of 100 mPas (cps) up to approx. 5 000 mPas (cps), tested according to as first alternative the European Pharmacopoeia and as second alternative the US Pharmacopoeia. In case of that tests are described in both pharmacopoeias, the method in the European one has prevalence.

The essential component, the high viscosity water soluble polymer, constitutes 51-100% w/w of the components forming the lag time controlling layer, i.e. after any solvents or dispersion/suspension media from the spraying solution/dispersion/suspension has been evaporated. Preferably the essential component constitutes 70-100% w/w of the lag time controlling layer, and more preferably the essential component constitutes 85-100% w/w of the lag time controlling layer.

In one alternative embodiment of the invention the lag time controlling layer comprises mixtures of high viscosity water soluble polymers.

In another alternative embodiment of the invention the lag time controlling layer only comprises high viscosity water soluble polymers of the same type but having different viscosities, disregarding trace amounts of solvents/water that may be remains from the coating process.

In a preferred alternative embodiment of the invention the lag time controlling layer comprises a moderately alkaline quality of one or more high viscosity water soluble polymer component, such as a moderately alkaline quality of HPMC or of HEC. With a moderately alkaline quality of a high viscosity water soluble polymer means a quality that gives a pH when measured according to Pharmacopoeia Europa between 7.0-9.0. This feature gives stability advantages to the dosage form.

In a further alternative embodiment of the invention the lag time controlling layer only comprises a single high viscosity water soluble polymer, i.e. the essential component constitutes 100% w/w of the lag time controlling layer, disregarding trace amounts of solvents/water that may be remains from the coating process. With a single polymer in this aspect, is considered a single polymer product, normally containing a limited range of polymer chain lengths distributed around an average value.

The total amount of lag time controlling layer applied onto the delay release modifying layered cores is chosen to effectuate the desired lag time by testing the in-vitro dissolution.

The dosage forms of the invention are having one portion of the PPI with a lag time in the range of 1-10 hours preferably 1-8 hours or most preferably 1-6 hrs. In an alternative embodiment, the dosage forms of the invention are having one portion of the PPI with a lag time in the range of 2-10 hours, preferably 2-8 hours or most preferably 2-6 hours. In a further alternative embodiment, the dosage forms of the invention are having one portion of the PPI with a lag time in the range of 4-10 hours, preferably 4-8 hours or most preferably 4-6 hours.

The man skilled in the art understands the lag time can be controlled by the amount and viscosity of the water soluble polymer in the lag time controlling layer, such that an increase of both these variables results in an increase in lag time. He will also know that extensive lag times, i.e. longer than 10-12 hrs, not are interesting to achieve, as formulations are excreated from the human body with time, and that the benefit of therapy regimens longer than once daily is questionable. The illustrating examples of this invention gives some formulas for lag time controlling layer application, which are easily modified by the man skilled in the art if so desired.

A group of preferred water soluble polymers are cellulose derivatives, e g HPMC (hydroxypropyl methylcellulose), HEC (hydroxyethyl cellulose), HPC (hydroxypropyl cellulose) and other polysaccharides such as pectin and pectinates (e.g. calcium pectinate), locust bean gum, tragacanth gum, guar gum, gum arabic, tamarind gum, tara gum, carrageenan, water-soluble alginates, pullulan and synthetic polymers such as polyethyleneoxides, polyoxyethylene-polyoxypropylene copolymers (Pluronics®), or a mixture thereof. HEC polymers to be included in the invention also includes such viscosity grades when tested in 1% solution fullfills the above specified viscosity requirements for “high viscosity”. Non-limiting examples of such BEC grades are Natrosol 250 from Aqualon with the following type designations; HHX, HHR, H4R, HR, MHR, MR, KR, and GR.

Especially preferred high viscosity water-soluble polymers are polymers of the type HPMC, polyethyleneoxides, HEC, xanthan gums, guar gums, or mixtures thereof.

Most preferred high viscosity water soluble polymers are HPMC or HEC or mixtures thereof.

The lag time is adjusted with the type of polymer or mix of polymers, and amount of polymer or mix of polymers, used in the delayed release controlling layer. Also the ratio between mixed polymer components in this layer may be used to adjust the lag time.

Optional Layer Comprising 2^{nd} Portion of PPI for Tablet Cores

The previously described tablets having a lag time controlling layer, are as one alternative embodiment of the invention coated, e.g. sprayed, with a dispersion/solution/suspension comprising a second portion/dose of active substance, together with a water soluble binder, a disintegrant and optionally a surfactant. The coating is performed in a suitable coating apparatus, to obtain cores having a layer comprising a 2^{nd} portion of PPI, deposited on top of the lag time controlling layer, giving an immediate release pulse when the final preparation is administered.

This layer, comprising the 2^{nd} portion of the PPI, comprises besides the active ingredient, a binder and optionally other excipients like disintengrants and alkaline pH-adjusting compounds.

Enteric Coating Layer(s) and Separating Layer(s).

Before applying an enteric coating layer onto the layered tablets, they may optionally be covered with one or more water soluble or in water rapidly disintegrating subcoating layers comprising pharmaceutical excipients optionally including alkaline compounds such as for instance pH-buffering compounds. This subcoating layer separates the composition of the layered tablets from the outer enteric coating layer.

The subcoating layer as well as the other type of layers, such as the lag time controlling layer, can be applied by coating or layering procedures in suitable equipments such as coating pan, coating granulator, centrifugal granulator or in a fluidized bed apparatus (including Wurster type) using water and/or organic solvents for the coating process. As an alternative the layer(s) can be applied by using powder coating technique.

Suitable materials for the optional separating layer are pharmaceutically acceptable compounds such as, for instance, sugar, polyethylene glycol, polyvinyl pyrrolidone, polyvinyl alcohol, polyvinyl acetate, hydroxypropyl cellulose, methylcellulose, ethylcellulose, hydroxypropyl methylcellulose, carboxymethylcellulose sodium and others, used alone or in mixtures. Additives such as plasticizers, colorants, pigments, fillers, ante tacking and antistatic agents, such as for instance magnesium stearate, titanium dioxide, talc, pH-buffering substances and other additives may also be included into the subcoating layer.

When the optional subcoating layer is applied to the coating layered tablets it may constitute a variable thickness. The maximum thickness of the optional subcoating layer is normally only limited by processing conditions. The subcoating layer may serve as a diffusion barrier and may act as a pH-buffering zone. The optional subcoating layer may improve the chemical stability of the active substance and/or the physical properties of the dosage form.

Finally the tablets having a lagtime controlling layer and optionally a subcoating layer are covered by one or more enteric coating layers by using a suitable coating technique. The enteric coating layer material may be dispersed or dissolved in either water or in suitable organic solvents. As enteric coating layer polymers one or more, separately or in combination, of the following can be used; e.g. solutions or dispersions of methacrylic acid copolymers, cellulose acetate phthalate, hydroxypropyl methylcellulose phthalate, hydroxypropyl methylcellulose acetate succinate, polyvinyl acetate phthalate, cellulose acetate trimellitate, carboxymethyl ethylcellulose, shellac or other suitable enteric coating layer polymer(s).

Additives such as dispersants, colorants, pigments, additional polymers e.g. poly(ethylacrylat, methylmethacrylat), antitacking and anti-foaming agents may also be included into the enteric coating layer. Other compounds may be added to increase film thickness and to decrease diffusion of acidic gastric juices into the acid susceptible material. The enteric coating layer(s) constitutes a thickness of approximately at least 10 μm, preferably more than 20 μm. The maximum thickness of the applied enteric coating layer(s) is nonnally only limited by processing conditions.

Any of the applied polymer containing layers, and specially the enteric coating layers may also contain pharmaceutically acceptable plasticizers to obtain desired mechanical properties. Such plasticizers are for instance, but not restricted to, triacetin, citric acid esters, phthalic acid esters, dibutyl sebacate, cetyl alcohol, polyethylene glycols, glycerol monoesters, polysorbates or other plasticizers. The amount of plasticizer is preferably optimized for each formula, in relation to the selected polymer(s), selected other additive(s) and the applied amount of said polymer(s).

In the alternative embodiment of the invention being enteric coated tablets that have no optional second drug comprising layer (giving an immediate release pulse when administered) under the enteric coating layer, such tablets are mixed with immediate release pellets or tablets (of suitable size) according to the art, and formulated into a capsule or a sachet. In such a way a final preparation giving both a delayed release pulse and an immediate relase pulse of the drug can be prepared.

Process

The final preparations of the present invention are made according to following principle process for the first alternative embodiment;

I) preparing a core material in the form of small tablets comprising an acid sensitive proton pump inhibitor as the only active drug;

II) coating the tablet cores obtained in step I) with a delay release modifying layer;

III) coating the delay release modifying layered tablet cores obtained from step II) with a lag time controlling layer comprising as essential component a high viscosity water soluble polymer;

IV) coating the lag-time controlling layered tablets obtained from step III) with an outer enteric coating, and an optional subcoating layer is applied before the enteric coating layer is applied;

V) incorporating the tablets product obtained in step IV) together with pellets having an outer enteric coating and an optional subcoating layer, giving immediate release of the PPI, into a capsule sachet, or multiple unit pellets system tablet.

The pellets giving immediate release are prepared according to the art, i.e. a core material comprising the PPI is layered with an enteric coating layer, and optional a subcoating layer is applied in between the core material and the enteric coating layer. These pellets giving an immediate release pulse is in one embodiment of the invention in the form of one or more tablet(s).

For the the other alternative embodiment the final preparations are made according to the following process;

I) preparing a core material in the form of small tablets comprising an acid sensitive proton pump inhibitor as the only active drug;

II) coating the tablet cores obtained from step I) with a delay release modifying layer;

III) coating the delay release modifying layered tablet cores obtained from step II) with a lag time controlling layer comprising as essential component a high viscosity water soluble polymer;

IV) coating the lag-time controlling layered tablets obtained from step III) with a layer comprising a 2^{nd} PPI portion;

V) optionally coating the tablets obtained from step IV) with an optional subcoating layer; and

VI) coating the tablets product obtained from step V) with an outer enteric coating;

VII) optionally formulating the enteric coated tablet(s) obtained from step VI) into a capsule, sachet or multiple unit pellets system tablet.

For step II, for both alternative embodiments above, when coating the cores obtained in step I), it is especially beneficial to use a composition that gives a delay release modifying layer that only is composed of the ingredients hydroxypropyl cellulose, talc and Mg-stearate, except any solvent/dispersant medial suspension media residues from the coating process.

For step III, for both alternative embodiments above, when coating the delay release modifying layered core from step II it is especially beneficial to utilize a dispersion of the high viscosity water soluble polymer prepared by

a) dispersing the high viscosity water soluble polymer in a non-solvent; and

b) adding an aqueous liquid or water to form a hydrated form of the dispersed polymer particles;

It should be understood that such a dispersed system can not be obtained by first dissolving the polymer in a water-containing liquid and then precipitating the system.

Lag Times

The embodiments are designed for having a lag time in the range of 1-10 hours, preferably in the range of 1-8 hours, and most preferably in the range of 1-6 hours.

As an alternative the embodiments are designed for having a lag time in the range of 2-10 hours, preferably in the range of 2-8 hours, and most preferably in the range of 2-6 hours.

As a further alternative the embodiments are designed for having a lag time in the range of 4-10 hours, preferably in the range of 4-8 hours, and most preferably in the range of 4-6 hours.

Final Dosage Forms

It is contemplated that the dosage forms of the invention before presentation to the patient is finalized to be in the form of capsules, sachets, multiple unit pellet system tablets or as as tablets comprising both an immediate and a delayed pulse. The finalized dosage form may comprise alternative combinations of tablets, other type of tablets and pellets, giving the delayed release pulse respectively the immediate release pulse. The delayed release pulse is according to this invention originating from tablets.

Tablets prepared according to the process description for “the other alternative embodiment” of the invention shortly described as having one PPI portion in the tablet core and a 2^{nd} PPI portion comprised in a coating layer, and as outer layer anenteric coating, are also contemplated to be the finalized dosage form as such.

The following combinations are contemplated;

| | Comprising | | |
|---|---|---|---|
| | Tablet(s) first | Tablets | |
| “Finalized” form | type | second type | Pellets |
| Capsule | Delayed rel. | Immediate | |
| | | rel. | |
| Capsule | Delayed rel. | | Immediate |
| | | | rel. |
| Capsule | Delayed rel. + | | |
| | Immediate rel. | | |
| Tablet (multiple | Delayed rel. | | Immediate |
| unit pellet system) | | | rel. |
| Sachet | Delayed rel. | Immediate | |
| | | rel. | |
| Sachet | Delayed rel. | | Immediate |
| | | | rel. |
| Sachet | Delayed rel. + | | |
| | Immediate rel. | | |
| The enteric coated tablet | Delayed rel. + | | |
| itself (comprising 2 PPI | Immediate rel. | | |
| portions) | | | |

### DEFINITIONS

Lag time/delay time: means for this invention that the dissolution of drug in vitro is delayed even after the enteric coated cores in form of pellets/tablets have been exposed for a first dissolution medium having pH 1.2 for 2 hours and then in a second dissolution medium having pH 6.8.

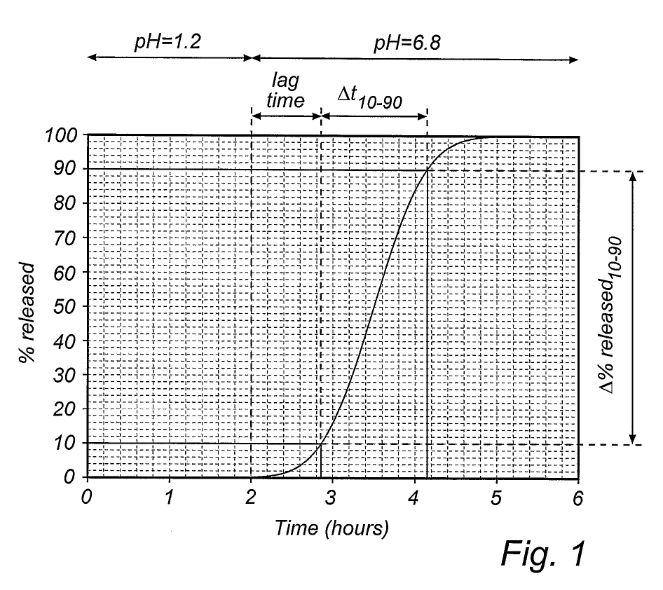

The lagtime is defmed as the time in the (second) dissolution medium required until 10% of the drug (of the dose in the delayed pulse) is released. For illustration, see FIG. 1.

The dissolution is determined in vitro using a USP dissolution Apparatus No. 2 with paddle, as described in USP XXI, page 1244, at 37° C., operated at 100 rpm and using 300 ml 0.1 N hydrochloric acid as first dissolution medium and then 1000 ml phosphate buffer pH 6.8 as second dissolution medium. The amount released is measured spectrophotometrically as the absorption obtained in % of the absorption of a reference omeprazole sample at the same wavelength (302 nm). For other PPI's the wavelength may be adjusted to a more suitable one (which can be determined by the man skilled in the art).

Steepness: the steepness is estimated as the average dissolution rate during the time elapsed between dissolution of 10% active drug until dissolution of 90% active drug (of the delayed dose). The drug release is measured and the steepness can e.g. be graphically evaluated after measurement.

The Steepness is defined as being the dissolved amount (80%) divided by the time in minutes required for dissolution of the 10-90% interval (of the delayed dose). This gives the Steepnes as the average rate during this period as being expressed in % per minutes ₍₁₀₋₉₀₎. For illustration, see FIG. 1.

The expression “negligible release” used in conjunction with the time period being the lag time, is less than 10% of the drug released.

The invention is illustrated by the following non limiting examples.

### EXAMPLES

### Example 1

Delayed Release Tablets Comprising 11 mg Esomeprazole Mg Trihydrate.

The schematic principle for the manufacture of the delayed release tablets was by making tablet cores comprising active ingredient (PPI) and coating them with layers in the following sequence; delay release modifying layer→lag time controlling layer→enteric coating layer.

Tablet cores were schematically made by granulating active substance together with excipients, drying and milling the obtained granules, mixing the granules with further additives and compressing the mixture.

Granulation:

| Excipients | Amount (g) |
|---|---|
| Esomeprazole-Mg trihydrate | 450 |
| Mannitol | 349 |
| Microcrystalline cellulose | 188 |
| Sodium starch glycolate | 60 |
| Hydroxypropyl methylcellulose (HPMC) 6 cps | 60 |
| Water | 350 |

The dry ingredients were mixed in an intensive mixer, Diosna Phamia P ⅙, for two minutes and thereafter the water was added during 2.5 minutes. Wet massing was continued for half a minute.

The obtained wet mass was put on trays and dried at 50° C. in a drying oven over night. The granules obtained were milled to pass a screen having 1.0 mm openings.

Mixing Before Compression

| Ingredients | Amount (g) |
|---|---|
| Esomeprazole granules | 900 |
| Talc | 41.5 |
| Microcrystalline cellulose | 51.5 |
| Sodium stearyl fumarate | 20.8 |

The granules and talc were mixed with the microcrystalline cellulose in a Kenwood mixer for 5 minutes in a Kenwood mixer at lowest mixing speed. Thereafter the sodium stearyl fumarate was added and the mixing was continued for another 2 minutes at the same speed.

Compression to Tablets:

The mixture was compressed in a rotary press, Korsch 106, equipped with punches giving round, size 4 mm in diameter, tablets having an average weight of 31 mg.

Delay Release Modifying Layer Application

| | |
|---|---|
| Ingredients | Amount (g) |

Core material

| | |
|---|---|
| Esomeprazole tablets | 150 |

Coating suspension

| | |
|---|---|
| Talc | 33.8 |
| Hydroxypropyl cellulose (75-150 cps) | 9.0 |
| Mg Stearate | 2.3 |
| water | 315 |

The hydroxypropyl cellulose was dissolved in the water. Thereafter the talc and the mg-stearate were suspended therein.

The coating was performed in a fluidized bed equipment, operating according to the Wurster principle, equipped with a liquid nozzle having a 0.8 mm in diameter opening. Inlet air temperature was 75° C., fluidizing air flow 40 Nm³/h, atomizer air pressure 2.0 bar, atomizer air flow 2.2 Nm³/h, spraying rate was 8-11 g/min resulting in an outlet air temperature of approx. 45° C.

Lag Time Controlling Layer Application

| | |
|---|---|
| Ingredients | Amount (g) |

Core material

| | |
|---|---|
| Tablets coated with delay release modifying | 150 |
| layer | |

Coating suspension

| | |
|---|---|
| Hydroxypropyl methyl cellulose 4000 cps* | 66.7 |
| Hydroxypropyl methyl cellulose 6 cps | 9.2 |
| Ethanol 99.5% | 1125 |
| Water | 143.3 |

*pH tested acc. To Pharm. Eur. to be 7.5

The high viscosity HPMC (the 4000 cps quality) powder was suspended in the ethanol (nonsolvent) while stirring. Under continued stirring a solution of the HPMC 6 cps and the water was gradually added, to result in low viscosity fluid comprising 75.9 g HPMC (polymer) per 1344.2 g total weight low viscosity fluid, i.e. a concentration of 5.7% (w/w).

The coating was performed in a fluidized bed equipment, operating according to the Wurster principle, equipped with a liquid nozzle having a 0.8 mm in diameter opening.

Inlet air temperature was 40° C., fluidizing air flow 40 Nm³/h, atomizer air pressure 2.5 bar, atomizer air flow 2.6 Nn/h, spraying rate was 15-16 g/min resulting in an outlet air temperature of approx. 20° C.

Enteric Coating Layer Application

| | |
|---|---|
| Ingredients | Amount (g) |

Core material

| | |
|---|---|
| Lag time layer coated tablets | 150 |

Coating suspension

| | |
|---|---|
| Methacrylic acid copolymer, type C, | 75 |
| 30% dispersion | |
| Talc | 4 |
| Triethyl citrate | 2.3 |
| Water | 94.5 |

First the triethyl citrate was dissolved in the water while stirring. Under continued stirring the polymer dispersion was gradually added, and finally the talc was suspended in the dispersion.

The coating was performed in the same coating equipment as the preceeding step. Inlet air temperature was 65° C., fluidizing air flow approx. 40 m³/h, atomizer air pressure 2.5 bar, atomizer air flow 2.6 Nm³/h, spraying rate was 6-7 g/min resulting in an outlet air temperature of approx. 38° C.

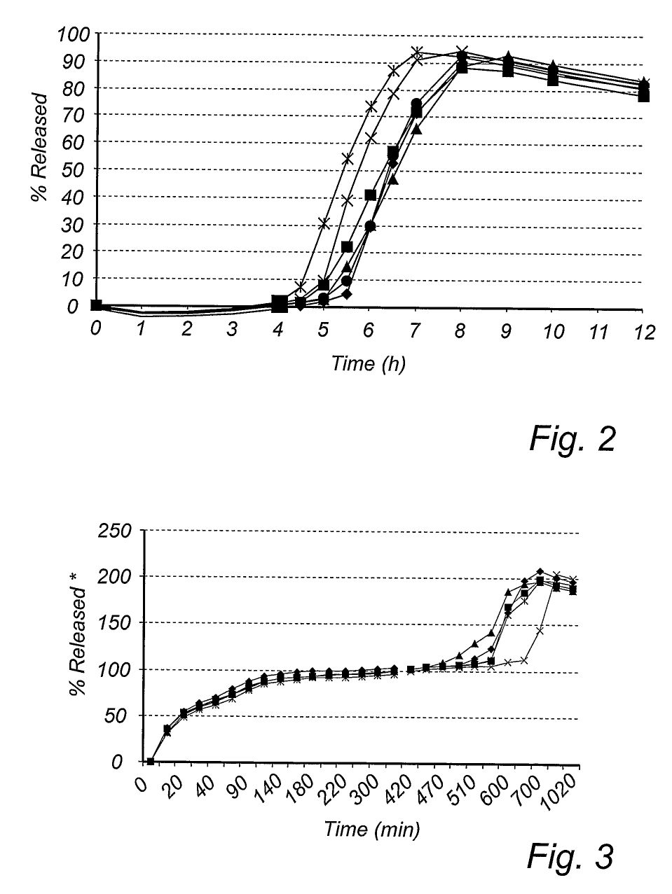

A sample of the obtained prodct was tested for in vitro dissolution. The dissolution profile obtained is presented in FIG. 2.

The dissolution test was made in USP dissolution apparatus No. 2 equipped with paddle and stationary basket. The paddle was operated at 75 rpm. As dissolution media was used in the 2 hrs pre-exposure phase 300 ml 0.1 M HCl (37° C.), and then the medium was changed to 1000 ml phosphate buffer pH 6.8 (37° C.).

Amount released esomeprazole magnesium was measured by UV-spectroscopy at 302 nm. The declining end phase of some of the release curves (absorption value curve) may be attributed to some degradation in the dissolution medium.

The lag time evaluated is approx. 5 hours.

### Example 2

Capsule Showing an Immediate Release Pulse and a Delayed Release Pulse of Esomeprazole Magnesium (40 mg+11 mg).

The schematic principle for the manufacture of the biphasic pulsed release capsules was by filling both pellets with immediate release and a tablet with delayed release (i.e. a tablet having the combined subcoat and lag time controlling layers according to the invention) into a hard gelatine capsule.

I.e. the following sequence was followed;

preparing delayed release tablets (lag time pellets according to the invention) as described in Example 1→filling a tablet into a capsule→filling the obtained tablet comprising capsule with immediat release pellets.

| Ingredients | Amount/capsule | |
|---|---|---|
| Delayed Release tablet (from Exemple 1) | 62 | mg (1 tablet) |
| Immediate release pellets (from Nexium ® capsule) | 171 | mg |
| Hard gelatin capsule (Size 2) | 1 | piece |

### Example 3

Enteric coated tablet showing an immediate release pulse and a delayed release pulse of esomeprazole magnesium (10 mg+11 mg).

The schematic principle for the manufacture of the delayed and immediate release tablets is to start with the tablets having cores comprising PPI, delay release modifying layer and lag time controlling layer produced according to example 1, and coating them with layers in the following sequence; layer comprising 2^{nd} PPI portion (giving immediate relase)→subcoating layer→enteric coating layer.

The tablets are coated in the same fluidized bed equipment as described in Example 1, operating according to the Wurster principle, equipped with a liquid nozzle having a 0.8 mm in diameter opening. Inlet air temperature is set to 80° C., fluidizing air flow approx. 45 Nm³/h, atomizer air pressure 2.8 bar, atomizer air flow 2.8 Nd/h, spraying rate is 6-11 g/min.

Application of Layer Comprising 2^{nd} PPI Portion;

| | |
|---|---|
| Ingredients | Amount (g) |

Core material

| | |
|---|---|
| Lag time layer coated tablets from Ex. 1 | 216 |
| (approx. 54 mg each) | |

Coating suspension

| | |
|---|---|
| Esomeprazole-Mg trihydrate | 61.1 |
| Polysorbate 80 | 1.2 |
| Hydroxypropyl methyl cellulose 6 cps | 9.1 |
| Croscarmellose sodium | 4 |
| Water | 373 |
| Suspension weight: | 448.4 |

The coating is continued until the avrage tablet weight has increased with 13 mg.

Application of Subcoating

| | |
|---|---|
| Ingredients | Amount (g) |

Core material

| | |
|---|---|
| Tablets from above | 268 |

Coating suspension

| | |
|---|---|
| Talc powder | 49.2 |
| Hydroxypropyl cellulose (75-150 cps) | 13.6 |
| Mg-Stearate | 3.2 |
| Water | 480 |
| Suspension weight: | 546 |

The hydroxypropyl cellulose is dissolved in the water. Thereafter the talc and the Mg stearate is suspended therein. The coating is performed in the same equipment as for the preceeding step, operating according to the Wurster principle, equipped with a liquid nozzle having a 0.8 mm in diameter opening. Inlet air temperature is set to 75° C., fluidizing air flow approx. 45 Nm³/h, atomizer air pressure 2.8 bar, atomizer air flow 2.8 Nm³/h, spraying rate is 6-11 g/min.

The coating is continued until the average tablet weight has increased with 12-14 mg.

Application of Enteric Coating Layer;

| | |
|---|---|
| Ingredients | Amount (g) |

Core material

| | |
|---|---|
| Subcoated tablets from above | 240 |

Coating suspension

| | |
|---|---|
| Methacrylic acid copolymer, type C, | 156 |
| 30% dispersion | |
| Talc | 8.3 |
| Triethyl citrate | 4.8 |
| Water | 196 |

First the triethyl citrate is dissolved in the water while stirring. Under continued stirring the polymer dispersion is gradually added, and finally the talc is suspended in the dispersion.

The coating is performed in the same coating equipment as the preceeding step. Inlet air temperature is 65° C., fluidizing air flow approx. 40 m³/h, atomizer air pressure 2.5 bar, atomizer air flow 2.6 Nm³/h, spraying rate is 6-7 g/min.

The coating is continued until the average tablet weight has increased with approx. 16 mg.

### Example 4

Delayed release tablets (for subsequent enteric coating) showing an immediate release pulse of lansoprazole (10 mg) and a delayed release pulse of esomeprazole magnesium (10 mg).

The schematic principle for the manufacture of the delayed release tablets is to start with the tablets having cores according to example 1, and coating them with layers in the following sequence;→delay release modifying layer→lag time controlling layer→layer comprising 2^{nd} PPI portion (giving immediate relase).

It is to be noted that the tablets according to this example not are enteric coated. They may later be enteric coated acccording to previous examples, like in Example 1, to obtain an embodiment of the claimed invention.

Application of Delay Release Modifying Layer

| | |
|---|---|
| Ingredients | Amount (g) |

Core material

| | |
|---|---|
| Esomeprazole tablet cores (acc. to Example 1) | 150 |

Coating suspension

| | |
|---|---|
| Talc | 33.8 |
| Hydroxypropyl cellulose (75-150 cps) | 9.0 |
| Sodium Stearyl Fumarate | 2.3 |
| Water | 315 |

The hydroxypropyl cellulose was dissolved in the water. Thereafter the talc and the sodium stearyl fumarate were suspended therein.

The coating was performed in a fluidized bed equipment, operating according to the Wurster principle, equipped with a liquid nozzle having a 0.8 mm in diameter opening. Inlet air temperature was 65° C., fluidizing air flow 60 Nm³/h, atomizer air pressure 1.5 bar, atomizer air flow 1.6 Nm³/h, spraying rate was 8-11 g/min resulting in an outlet air temperature of approx. 50° C.

Application of Lag Time Controlling Layer;

| | |
|---|---|
| Ingredients | Amount (g) |

Core material

| | |
|---|---|
| Tablets coated with delay release modifying | 150 |
| layer (from above) | |

Coating suspension

| | |
|---|---|
| Hydroxyethyl cellulose 250 HHX | 70 |
| Hydroxyethyl cellulose PLUS 330CS | 7.5 |
| Ethanol 99.5% | 751 |
| Water | 183 |

The Hydroxyethyl cellulose (sieved to pass 125 μm) was suspended in the ethanol (non solvent) while stirring. Under continued stirring water was gradually added, to result in low viscosity fluid comprising 77.5 g Hydroxyethyl cellulose (polymer) per 1012 g total weight low viscosity fluid, i.e. a concentration of 7.7% (w/w).

The coating was performed in a fluidized bed equipment, operating according to the Wurster principle, equipped with a liquid nozzle having a 0.8 mm in diameter opening. Inlet air temperature was 42° C., fluidizing air flow 60 Nm³/h, atomizer air pressure 1.5 bar, atomizer air flow 1.6 Nm³/h, spraying rate was 11-14 g/min resulting in an outlet air temperature of approx. 32° C.

Application of Layer Comprising 2^{nd} PPI Portion;

| | |
|---|---|
| Ingredients | Amount (g) |

Core material

| | |
|---|---|
| Lag time layer coated tablets (from above) | 150 |

Coating suspension

| | |
|---|---|
| Lansoprazole | 29.4 |
| Polysorbate 80 | 0.6 |
| Hydroxypropyl methyl cellulose 6 cps | 4.4 |
| Croscarmellose sodium | 5.1 |
| Water | 498 |

The coating was performed in a fluidized bed equipment, operating according to the Wurster principle, equipped with a liquid nozzle having a 0.8 mm in diameter opening. Inlet air temperature is set to 65° C., fluidizing air flow approx. 60 Nm³/h, atomizer air pressure 1.6 bar, atomizer air flow 1.6 Nm³/h, spraying rate is 11-14 g/min resulting in an outlet air temperature of approx. 45° C.

Samples of the obtained product were tested for in vitro dissolution. The dissolution obtained is presented in FIG. 3.

The dissolution test was made in USP dissolution apparatus No. 2 equipped with paddle and stationary basket. The paddle was operated at 75 rpm. Dissolution media was 1000 ml phosphate buffer pH 6.8 (37° C.). No preexposure to HCl was done for these samples.

Amount released lansoprazole was measured by UV-spectroscopy at 285 nm (time phase 0-360 minutes). Amount released esomeprazole magnesium was measured by UV-spectroscopy at 302 nm (time phase 420-1020 minutes).

The lagtime evaluated is approx. 10 hours.

1. An oral solid pharmaceutical dosage form comprising;
(i) an acid sensitive proton pump inhibitor (PPI) as the sole active ingredient and,
(ii) a core material containing the PPI and in the form of a mixture of tablets and pellets,
wherein:
(A) the tablets give a delayed release pulse of the PPI, wherein the tablets have the following sequence of layers on the core material:
A1—a delay release modifying layer;
A2—a lag time controlling layer comprising a high viscosity water soluble polymer;
A3—an optional subcoating layer; and
A4—an outer enteric coating layer; and
(B) the pellets give an immediate release pulse of the PPI, wherein the pellets have the following sequence of layer(s) on the core material:
B1—an optional subcoating layer; and
B2—an outer enteric coating layer.

2. An oral solid pharmaceutical dosage form comprising:
(i) an acid sensitive proton pump inhibitor (PPI) as the sole active ingredient, and
(ii) a core material in the form of tablets and containing the PPI,
wherein each tablet gives a delayed release pulse of the PPI and an immediate release pulse of the PPI and has the following sequence of layers on the core material:
a—a delay release modifying layer;
m—a lag time controlling layer comprising a high viscosity water soluble polymer;
(c)—a layer comprising the second portion of the PPI;
d—an optional subcoating layer; and
(e)—an outer enteric coating layer.

3. The oral pharmaceutical dosage form according to claim 1 or 2, wherein the dosage form is a capsule.

4. The oral pharmaceutical dosage form according to claim 1 or 2, wherein the dosage form is a sachet.

5. The oral pharmaceutical dosage form according to claim 1 wherein the pellets giving an immediate release pulse are in the form of one or more tablet(s).

6. The oral pharmaceutical dosage form according to claim 1 or 2, wherein the acid sensitive proton pump inhibitor is an alkaline salt of esomeprazole.

7. The oral pharmaceutical dosage form according to claim 1 or 2, wherein the acid sensitive proton pump inhibitor is esomeprazole magnesium.

8. The oral pharmaceutical dosage form according to claim 1 or 2, wherein the acid sensitive proton pump inhibitor is omeprazole magnesium.

9. The oral pharmaceutical dosage form according to claim 1 or 2, wherein the delayed release pulse has a lag time in the range of 1-10 hours.

10. The oral pharmaceutical dosage form according to claim 9, wherein the lag time is in the range of 2-8 hours.

11. The oral pharmaceutical dosage form according to claim 1 or 2, wherein the lag time controlling layer consists essentially of a high viscosity water soluble polymer.

12. The oral pharmaceutical dosage form according to claim 1 or 2, wherein the water soluble polymer in the lag time controlling layer is a high viscosity hydroxypropyl methyl cellulose or a high viscosity hydroxyethyl cellulose.

13. The oral pharmaceutical dosage form according to claim 12, wherein the high viscosity hydroxypropyl methyl cellulose or hydroxyethyl cellulose gives a pH of between 7.0-9.0 when measured according to Pharmacopoeia Europa.

14. The oral pharmaceutical dosage form according to claim 1 or 2 wherein the delay release modifying layer comprises one or more water soluble polymer(s), talc and a hydrophobizing agent selected from the group consisting of Mg-stearate, glyceryl behenate, and sodium stearyl fumarate.

15. The oral pharmaceutical dosage form according to claim 1 or 2 wherein the delay release modifying layer consists essentially of hydroxypropyl cellulose, talc and Mg-Stearate.

16. A process for preparing an oral pharmaceutical dosage form according to claim 1, wherein the dosage form comprises (i) an acid sensitive proton pimp inhibitor (PPI) as the sole active ingredient and (ii) a core in the form of a mixture of tablets and pellets and containing the PPI, the process comprising the following steps:
(a) preparing tablets containing the PPI;
(b) coating the tablets obtained in step (a) with a delay release modifying layer;
(c) coating the tablets obtained in step (b) with a lag time controlling layer comprising a high viscosity water soluble polymer;
(d) optionally applying a subcoating layer to the tablets obtained in step (c);
(e) coating the tablets obtained in step (c) or (d) with an outer enteric coating to obtain tablets giving a delayed release pulse of the PPI;
(f) mixing the tablets obtained in step (e) with pellets containing the PPI and having an outer enteric coating and an optional subcoating layer, wherein the pellets give an immediate release of the PPI; and
(g) formulating the mixture of tablets and pellets from step (f) into the dosage form.

17. A process for preparing an oral pharmaceutical dosage form according to claim 2, wherein the dosage form comprises (i) an acid sensitive proton pump inhibitor (PPI) as the sole active ingredient and (ii) a core material in the form of tablets and containing the PPI, the process comprises the following steps:
(a) preparing tablets with a first portion of the PPI;
(b) coating the tablets obtained in step (a) with a delay release modifying layer;
(c) coating the tablets obtained in step (b) with a lag time controlling layer comprising a high viscosity water soluble polymer;
(d) coating the tablets obtained in step (c) with a layer comprising a second portion of the PPI;
(e) optionally coating the tablets obtained in step (d) with a subcoating layer;
(f) coating the tablets obtained in step (d) or (e) with an outer enteric coating; and
(g) formulating the enteric coated tablets obtained in step (f) into the dosage form.

18. The process according to claim 16, wherein the pellets giving an immediate release pulse are in the form of one or more tablet(s).

19. The process according to claim 16 or 17, wherein the step of coating the tablet cores with the lag time controlling layer is performed by applying a dispersion of the high viscosity water soluble polymer prepared by the steps of:
a) dispersing the high viscosity water soluble polymer in a non-solvent; and
b) adding an aqueous liquid or water to form a hydrated form of the dispersed polymer particles.

20. The process according to claim 16 or 17, wherein the delay release modifying layer consists essentially of hydroxypropyl cellulose, talc and Mg-Stearate.

21. The process according to claim 16 or 17, wherein the delayed release pulse has a lag time in the range of 1-10 hours.

22. A method for improving inhibition of gastric acid secretion, the method comprising administering an oral pharmaceutical dosage form as defined in any one of claims 1 or 2 to a patient in need thereof.

23. (canceled)

24. The oral pharmaceutical dosage form according to claim 1 or 2, wherein the tablets have a diameter of less than or equal to 5 mm.

25. The process according to claim 16 or 17, wherein the tablets have a diameter of less than or equal to 5 mm.

26. The process according to claim 16 or 17, wherein the dosage form is a capsule, sachet, or multiple unit pellets system tablet.
